(19) United States
(12) Patent Application Publication
(10) Pub. No.: US 2007/0282329 A1
(43) Pub. Date: Dec. 6, 2007

(54) Bipolar high-frequency incision tool for an endoscope

(75) Inventor: Tomohiro Kawano, Saitama-ken (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 11/442,422

(22) Filed: May 30, 2006

(51) Int. Cl. A61B 18/14

(52) U.S. Cl. 606/48

(57) ABSTRACT

A bipolar high-frequency incision tool for an endoscope is provided having a flexible insertion portion configured to be inserted into a body cavity. First and second elongated electrodes are mounted to a distal end of the insertion portion so as to be movable between an open position and a closed position. The first and second electrodes are connectable with conductive wires that provide a high frequency voltage, and have corresponding first and second surfaces. The first and second surfaces are spaced apart from each other when the first and second electrodes are in an open position, and the first and second electrodes remain spaced from one another when the first and second electrodes are moved to the closed position. In addition, the first and second surfaces each have inwardly tapered side surfaces configured to form respective narrowed blade edges.

### FIELD OF THE INVENTION

The present invention relates to a bipolar high-frequency incision tool for an endoscope, and more particularly to an incision tool for an endoscope having increased current density.

### BACKGROUND AND MATERIAL INFORMATION

It is known in the art to provide a high frequency incision instrument, which utilizes a needle or generally rod-shaped electrode in combination with a large counter electrode placed on the body surface of the patient. High frequency electric current is generated between the needle or rod-shaped electrode and the counter electrode to cauterize the tissue in the vicinity of the needle or rod-shaped electrode. Further, because the high frequency incision instrument, of the rod or needle type, is monopolar there is a large heat impact to a surrounding area of tissue.

The disadvantage of the prior art high frequency incision instrument is that it may make a hole in the tissue that is much deeper than required if the high frequency current is generated continuously for a long period of time since the current flows from one electrode located inside the human body to the other electrode placed on the outer body surface.

Forming such a deep hole can be avoided by generating the current intermittently; however, this requires longer surgery.

Thus, there is a need for a bipolar high-frequency incision instrument for an endoscope that can form an appropriate incision during a relatively short surgery time without the danger of cutting deeply into an affected area.

### SUMMARY OF THE INVENTION

A non-limiting embodiment of the present invention provides a bipolar high-frequency incision tool having a flexible insertion portion configured to be inserted into a body cavity through an endoscope. The bipolar high-frequency incision tool has first and second elongated electrodes mounted to a distal end of an insertion portion so as to be movable between an open position and a closed position. The first and second electrodes are connected to conductive wires that provide a high frequency voltage, and have corresponding first and second surfaces, respectively. The first and second surfaces are spaced apart from each other when the first and second electrodes are in an open position, and the first and second electrodes remain spaced from each other when the first and second electrodes are moved to the closed position. In addition, the first and second surfaces each have inwardly tapered side surfaces configured to form respective blade edges. Further, the configuration of the blades increases the current density at the blade edges.

Another feature includes the first and second surfaces may have a generally uniform spacing along a length of the first and second surfaces when the first and second electrodes are in a closed position.

According to another feature, the blade edges may be shorter in length than the corresponding first and second surfaces, and in a further feature, the blade edges may be configured to cut an affected area. In addition the first and second electrodes may move between the open and closed positions along a common plane.

Further, according to another feature, the blades may be provided having a generally triangular cross-section. Moreover, the first and second electrodes may have a generally triangular cross section in the area formed by the tapered side surfaces, and the blade edges may be formed by a longitudinally extending protrusion portion provided at the apex thereof. Additionally, the protruding portion may have a generally rounded outer surface extending along its length.

In a further aspect of the invention, a tool for an endoscope is provided that includes a supporting member configured to be connected to an insertion portion of the endoscope, first and second jaws pivotally connected to the supporting member and connectable to operating wires that are operable to move the jaws between open and closed positions, the jaws each having first and second surfaces, wherein the first and second surfaces each have inwardly tapered side surfaces configured to form respective blade edges that are narrower in width than the width of the jaws, and wherein the first and second jaws remain laterally spaced from each other when the first and second are moved to the closed position. The first and second surfaces may have a generally uniform spacing along a length of the first and second surfaces when the first and second jaws are in a closed position. The blade edges may be configured to increase current density at the edges thereof.

In other aspects, the blade edges may be shorter in length than the first and second surfaces. Also, the blade edges may be configured to cut an affected area. Moreover, the first and second jaws may be movable between the open and closed positions along a common plane. Additionally, the first and second jaws may have a generally triangular cross section in the area formed by the tapered side surfaces, and each blade edge may be formed by a longitudinally extending protruding portion provided at the apex thereof. Further, the protruding portion may have a generally rounded outer surface extending along its length.

### BRIEF DESCRIPTION OF THE DRAWINGS

The present invention is further described in the detail description which follows, in reference to the noted plurality of drawings, by way of non-limiting examples of preferred embodiments of the present invention, in which like characters represent like elements throughout the several views of the drawings, and wherein:

FIG. 1 shows a side view of a bipolar high-frequency incision tool in a closed position according to an embodiment of the invention;

FIG. 2 shows a front end view of FIG. 1;

FIG. 3 shows a top plan view of the bipolar high-frequency incision tool of FIG. 1 shown connected to operating wires surrounded by an insulating tube and sheath;

FIG. 4 shows a cross-section of the bipolar high-frequency incision tool of FIG. 3 taken along line A-A;

FIG. 5 shows a perspective view of an affected area and the bipolar high-frequency incision tool in an open position;

FIG. 6 shows a perspective view of the affected area and an incision being formed by the bipolar high-frequency incision tool when in a closed potion;

FIG. 7 shows a perspective view of the insulating block of the bipolar high-frequency incision tool; and

FIG. 8 shows a sectional view of the insulating block of the bipolar high-frequency incision tool.

### DETAILED DESCRIPTION OF THE DRAWINGS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

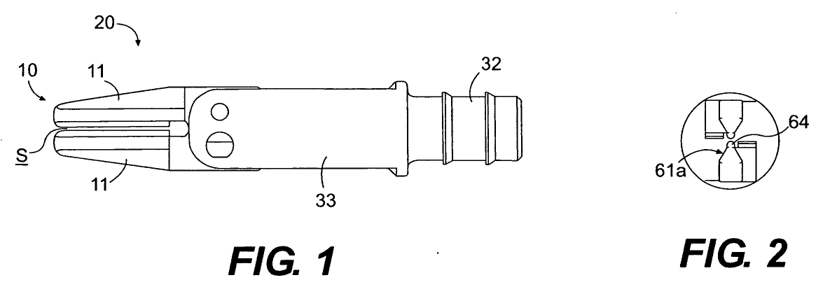

Referring to the drawings, wherein like characters represent like elements, FIG. 1 shows a perspective view of a bipolar high-frequency incision tool 10 for an endoscope according to a non-limiting embodiment of the present invention. The tool may be used in conjunction with a bipolar high-frequency endoscopic surgical system described, for example, in U.S. Pat. No. 6,969,389 and U.S. Patent Publication No. 2003/0191465, both disclosures being expressly incorporated herein by reference in their entireties.

FIG. 1 schematically shows a side view of a bipolar high-frequency incision tool 10 for an endoscope according to a first embodiment of the invention. The bipolar high-frequency incision tool 10 is connectable to a high frequency power supply (not shown).

The bipolar high-frequency incision tool 10 includes an operation portion (not shown) and an insertion portion 20 connected to a distal end of the operation portion.

The insertion portion 20 is configured to be introduced into a body cavity through a treatment tool insertion channel of an endoscope (not shown). The insertion portion 20 includes an elongated and flexible sheath 30, a pair of conductive wires 31, as shown in FIG. 4, slidably inserted through the sheath 30, and a pair of electrodes 11 (or jaws) provided at the distal end of the insertion portion 20 and connected to the conductive wires 31. The sheath 30 is preferably made of any suitable insulating material, such as poly-tetra-fluoro-ethylene (PTFE).

The conductive wires 31 may be detachable connected to a high frequency power supply (not shown). In this regard, one of the conductive wires 31 is connected to a positive terminal of the power supply and the other to the negative terminal, thereby providing the bipolar high-frequency incision tool 10.

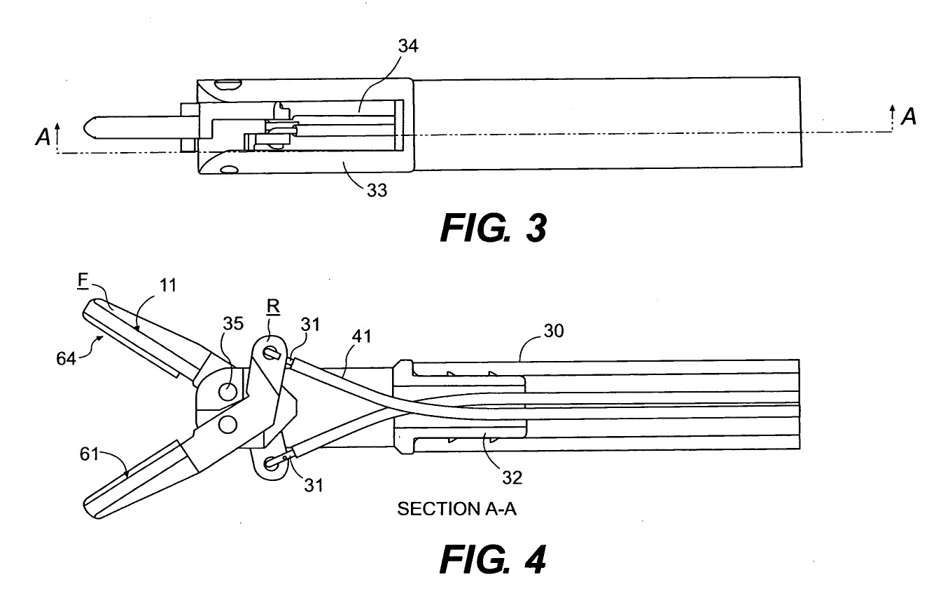

FIG. 4 shows a sectional side view of the distal end portion of the bipolar high-frequency incision tool 10 shown in FIG. 3. Note that the pair of electrodes 11 is shown in a closed position in FIG. 1, and at an open position in FIG. 4.

As shown in FIGS. 1, 3 and 4, a supporting member 32 for supporting the pair of electrodes 11 is mounted to the distal end of the flexible sheath 30. The supporting member 32 may be made of any suitable hard insulating material, such as rigid synthetic plastic material. The supporting member 32 has two arms 33 extending in a forward direction and parallel to each other to form a slit 34 having a generally uniform width. Two pins 35 are supported between the arms 33 in the vicinity of the distal end thereof. The pins 35 are arranged generally parallel to and spaced apart from each other, and generally perpendicular to side walls of the slit 34. The pins 35 may be made of any suitable material, such as stainless steel.

The pair of electrodes 11 is partially provided within the slit 34 of the supporting member 32 and each is rotatably mounted to a respective one of the pair of pins 35. Thus, the pair of electrodes 11 can move between the closed position shown in FIG. 1, at which the electrodes 11 remain slightly spaced from each other, and the open position shown in FIG. 4 in which the electrodes 11 are located further apart from each other.

The rear ends or proximal ends of the electrodes 11 are connected with the conductive wires 31. Each of the conductive wires 31 is covered with an insulating tube 41 except the end portion thereof at which the conductive wire 31 is connected to the corresponding electrode 11.

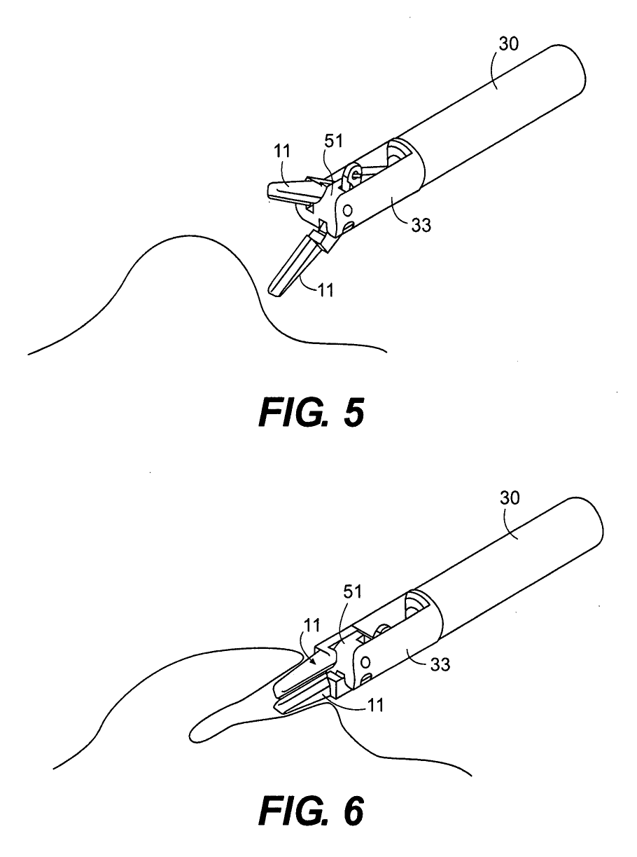

An insulating block 51, as shown in FIGS. 5, 7 and 8, is provided on the outer end of the supporting member 32 to prevent the electrodes 11 from coming into contact to each other within the slit 34. The insulating block 51 is located between the electrodes 11 and may be formed in one piece with the supporting member 32, or may be formed separately and supported by the pins 35. The insulating block may be made of resin, e.g., poly-tetra-fluoro-ethylene (PTFE).

FIG. 4 is a perspective view of the insertion portion 20. The electrodes 11 are generally elongated opposed members that may be made of any suitable electrode material, such as a metal, e.g., stainless steel. The electrodes 11 include a generally elongated front portion F (at a free end) and a generally elongated rear portion R (proximate wires 31). When the electrodes 11 are mounted to the supporting member 32, the front portion is located at a position forward of the arms 33, and the rear portion is positioned generally between the arms 33 (see FIG. 3).

Two through holes may be provided in the rear portion of each electrode 11. One through hole, a supporting through hole, is configured to be a supporting hole located generally at the center of each electrode 11. The other one is a connection hole provided in the vicinity of the rear portion of each electrode 11.

As shown in FIG. 4, each electrode 11 is pivotably mounted to the supporting member 32 by insertion the corresponding pins 35 through the respective supporting hole. Thus, each electrode 11 can swing between the closed position shown in FIG. 1 and the opened position shown in FIG. 4.

As can be seen in FIG. 4, the distal end of each conductive wire 31, which is exposed from the corresponding insulating tube 41, is passed through the respective connecting hole to be connected to respective electrodes 11.

The rear portion of each electrode 11 is slightly offset (FIG. 3) so that the conductive wires 31 that slide back and forth within the sheath 30 can swing the electrodes 11 around corresponding pin 35 between the open and closed positions.

The electrodes 11 have corresponding first and second surfaces (both labeled 61), respectively, each provided with a blade edge 64. In one embodiment, the electrodes 11 are configured such that when the electrodes are in the closed position (FIGS. 1, 3 and 6) the blade edges 64 remain slightly spaced, for example by about 0.05 mm to about 0.5 mm, which may result in less tissue damage. In addition, the surfaces 61 each may be provided with inwardly tapered side surfaces 61a configured to form the side surfaces of respective blade edges 64. Further, the narrowed configuration of the blade edges 64, formed due to the tapered side surfaces 61a, increases the current density at the blade edges 64.

Thus, it should be appreciated that the decrease in surface area of the blade edges 64, which are configured to contact an affected area (thereby increasing the current density), ensure the secure and safe resection of only a desired portion of the affected area.

The first and second surfaces 61 are also provided having a generally uniform spacing S (for example, about 05 mm to about 0.5 mm) along a length of the first and second surfaces 61 when the electrodes 11 are in a closed position, as shown in FIG. 1. In addition, the uniform spacing of the electrodes 11 and corresponding blade edge 64 allows an incision to be made while tissue of an affected area is being pinched, thereby making the incision tool substantially easier to operate.

The blade edges 64 may be provided on the electrodes 11 so that the blade edges 64 are shorter in length than the first and second surfaces 61, as shown in FIG. 1.

As shown in FIG. 2, the blade edges 64 may be formed as a generally rounded, longitudinally extending protruding portion provided at an apex of the generally triangular portion of each electrode formed by the tapered side surfaces 61a. Of course, the blade edges may be configured to have any suitable shape selected to form a particular surgery.

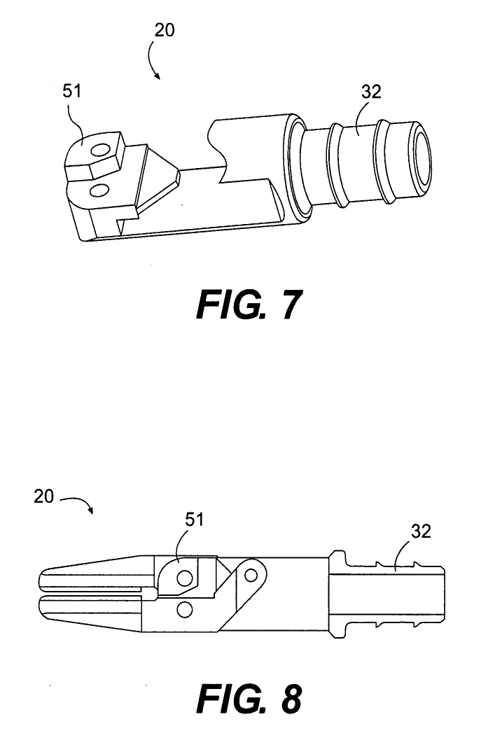

FIG. 7 shows the blade edges 64 forming an incision in an affected area. In addition the first and second electrodes 11 move between the open and closed positions along a common plane, shown in FIG. 2. In addition, the blades may be provided having a generally triangular cross-section.

It is further noted that the foregoing examples have been provided merely for the purpose of explanation and are in no way to be construed as limiting of the present invention. While the present invention has been described with reference to a preferred embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A bipolar high-frequency incision tool for an endoscope, comprising:
a flexible insertion portion configured to be inserted into a body cavity through an endoscope;
first and second elongated electrodes mountable to a distal end of said insertion portion so as to be movable between an open position and a closed position, said first and second electrodes being connectable with conductive wires that provide a high frequency voltage, said first and second electrodes having corresponding first and second surfaces, respectively;
wherein said first and second surfaces are spaced apart from each other when said first and second electrodes are in an open position, and said first and second electrodes remain spaced from each other when said first and second electrodes are moved to said closed position; and
wherein said first and second surfaces each have inwardly tapered side surfaces configured to form respective blade edges that are narrower in width than the width of said electrodes.

2. The bipolar high-frequency incision tool according to claim 1, wherein said first and second surfaces have a generally uniform spacing along a length of said first and second surfaces when said first and second electrodes are in a closed position.

3. The bipolar high-frequency incision tool according to claim 1, wherein said blade edges are configured to increase current density at said blade edges.

4. The bipolar high-frequency incision tool according to claim 1, wherein said blade edges are shorter in length than said first and second surfaces.

5. The bipolar high-frequency incision tool according to claim 1, wherein said blade edges are configured to cut an affected area.

6. The bipolar high-frequency incision tool according to claim 1, wherein said first and second electrodes are movable between said open and closed positions along a common plane.

7. The bipolar high-frequency incision tool according to claim 1, wherein said first and second electrodes have a generally triangular cross section in the area formed by said tapered side surfaces, and said blade edges are formed by a longitudinally extending protruding portion provided at the apex thereof.

8. The bipolar high-frequency incision tool according to claim 7, wherein said protruding portion has a generally rounded outer surface extending along its length.

9. A tool for an endoscope, comprising:
a supporting member configured to be connected to an insertion portion of the endoscope;
first and second jaws pivotally connected to the supporting member and connectable to operating wires that are operable to move the jaws between open and closed positions, said jaws each having first and second surfaces,
wherein said first and second surfaces each have inwardly tapered side surfaces configured to form respective blade edges that are narrower in width than the width of said jaws; and
wherein said first and second jaws remain laterally spaced from each other when said first and second jaws are moved to the closed position.

10. The tool according to claim 9, wherein said first and second surfaces have a generally uniform spacing along a length of said first and second surfaces when said first and second jaws are in a closed position.

11. The tool according to claim 9, wherein said blade edges are configured to increase current density at said blade edges.

12. The tool according to claim 9, wherein said blade edges are shorter in length than said first and second surfaces.

13. The tool according to claim 9, wherein said blade edges are configured to cut an affected area.

14. The tool according to claim 9, wherein said first and second jaws are movable between said open and closed positions along a common plane.

15. The tool according to claim 9 wherein said first and second jaws have a generally triangular cross section in the area formed by said tapered side surfaces, and said blade edges are formed by a longitudinally extending protruding portion provided at the apex thereof.

16. The bipolar high-frequency incision tool according to claim 15, wherein said protruding portion has a generally rounded outer surface extending along its length.
